(19) United States
(12) Patent Application Publication
(10) Pub. No.: US 2001/0047204 A1
(43) Pub. Date: Nov. 29, 2001

(54) Method for determination of a properly sized posterior chamber phakic refractive lens

(75) Inventors: Stephen Q. Zhou, Irvine, CA (US); George W. Rozakis, North Olmsted, OH (US)

Correspondence Address: Frost Brown Todd LLC, 2200 PNC Center, 201 East Fifth Street, Cincinnati, OH, 45202, US

(73) Assignee: Medennium, Inc.

(21) Appl. No.: 09/795,912

(22) Filed: Feb. 28, 2001

Related U.S. Application Data
(60) Provisional application No. 60/190,684, filed on Mar. 20, 2000.

(51) Int. Cl. A61F 2/16

(52) U.S. Cl. 623/6.11; 623/905

(57) ABSTRACT

A method for selecting the size of a phakic intraocular lens positioned in the eye between the iris and the natural lens, is disclosed. In this method, the white to white distance of the patient's eye is measured and the length of the lens is based on that measurement. A photographic method of measurement is disclosed.

This application is based on and claims priority from U.S. Provisional Application No. 60/190,687, filed Mar. 20, 2000.

### BACKGROUND OF THE INVENTION

The present invention relates to phakic refractive lenses implanted in the eye for the correction of ametropia. A posterior chamber phakic refractive lens (PRL) is surgically implanted behind the iris and in front of the human natural crystalline lens for correcting ametropia, such as myopia or hyperopia (FIG. 1). The PRL is the only reversible procedure for correcting severe refractive errors, both myopic and hyperopic patients. However, there are three major complications associated with PRL implantation. They are: (1) intraocular pressure (IOP) elevation; (2) cataract induction; (3) iris pigment dispersion. Only until all those three complications are successfully resolved will PRL technology become acceptable to surgeons and patients. Currently, IOP elevation has been successfully controlled by surgical iridotomy (i.e., two holes usually are made either by laser or knife). Cataract induction and iris pigment dispersion can be minimized or eliminated by using an anatomically designed PRL.

One of the anatomical designs for the PRL is the floating or no permanent fixation design. The PRL is loosely held in place behind the iris and in front of the human natural crystalline lens. The ideal situation is that the PRL should have a dimension approximately equal to or slightly smaller than the patient's eye size. However, individual patients vary in their eye sizes. A mismatch of a PRL's dimension with a patient's eye size will prevent the eye from utilizing the PRL's intended design features to their full extent. Furthermore, it may cause the PRL to fail in its basic design functions. Accordingly, PRL manufacturers must instruct ophthalmologists how to select a properly sized PRL for an individual patient.

The present invention aims to establish a method for selection of a properly sized PRL for an individual patient to ensure the full achievement of the PRL design features. It solves the problem of a mismatch between PRL dimensions and an individual's eye size.

There are a number of patents describing the PRL concept or some specific lens designs. U.S. Pat. No. 4,585,456, Blackmore (issued Apr. 29, 1986), discloses a phakic intraocular lens (IOL) composed of flexible materials positioned against the natural lens of the eye and being held in place immediately adjacent to the natural lens and the ciliary sulcus. It also discloses that surgeons need to select the proper optics for the eye. However, there are no disclosures of the phakic IOL's size, nor the method for selecting the proper size.

Fedorov has several US patents describing features of phakic refractive lenses which are said to avoid potential complications. In U.S. Pat. No. 5,480,428, issued Jan. 2, 1996, Fedorov discloses a novel phakic lens design which has a hole through the center of the optic body. This open hole will allow aqueous humor flow through the lens body, thereby preventing IOP elevation. Fedorov, U.S. Pat. No. 5,258,025, issued Nov. 2, 1993, discloses that post-operative inflammation, caused by the contacting of the supporting elements with the ocular tissue, is prevented by moving supporting elements to the periphery of the phakic lens. The diameter of the position elements ranges from about 10 mm to about 10.5 mm. The Zinn's zonules are said to be strong enough to hold the supporting elements in place without causing inflammation. Fedorov, U.S. Pat. No. 5,766,245, issued Jun. 16, 1998, discloses an IOL for correcting moderate to severe hypermetropia. The length of the IOL is from 10 to 13 mm. However, in none of the above Fedorov patents, is there any disclosure of a method for selection of a properly sized PRL for an individual patient. For example, for a specific individual patient, no guidance is given to assist the surgeon in picking a 10 mm lens or a 13 mm lens to achieve the proper fit in the eye.

Kelman, in U.S. Pat. No. 4,769,035, issued Sep. 6, 1988, discloses a surgical procedure for correction of the eyesight of a human eye by implanting an artificial lens between the iris and anterior surface of the human lens. It is a multi-step procedure including the following two steps. First, the patient's refractive error is measured so that the artificial lens can be properly selected with desirable optical power for the patient. Second, the shape of the anterior surface of the patient's natural lens is determined so that the artificial lens can be selected to have its posterior surface shape conforming to the anterior surface of the patient's natural lens. In other words, the posterior surface of the optic portion of the artificial lens is in substantial face-to-face contact with the anterior surface of the patient's natural lens. Kelman also points out that ultra-sonography technology (A scan or B scan) can be used for determining the shape of the patient's natural lens, and that the longitudinal length of the artificial lens is approximately 13 mm. Nevertheless, Kelman is silent on whether or how the length of the artificial lens needs to be varied for various patient eye sizes.

Lastly, Valunin's U.S. Pat. No. 6,015,435, issued Jan. 18, 2000, discloses a PRL and a method of fitting the PRL between the iris and the anterior surface of the human natural lens. The PRL's size and dimension are selected in such a way that the haptic bodies of the PRL do not contact the outermost circumference of the ciliary sulcus of the wearer at the same time. Among other disclosures, Valunin also indicates that the maximum diagonal haptic body dimension of the lens is from about 10.5 mm to about 11.5 mm (see FIG. 3).

Those skilled in the art understand that the sulcus-to-sulcus distance can be measured by modem imaging techniques, such as very high frequency ultrasound B-scan image. After the B-scan, the surgeon will be able to select a proper size for the patient. However, B-scan is time consuming and the instrument is costly. Therefore, an easy-to-measure and low cost alternative method for properly sizing the PRL is highly desirable. Accordingly, there is a great need in establishing a method for the selection of a PRL of any designs with proper size for any individual patient based on the patient's eye size. The ideal method will be simple, easy, accurate and low cost.

### SUMMARY OF THE INVENTION

The present invention relates to a method for selecting a phakic intraocular lens for a given patient comprising the steps of:

(a) measuring the white-to-white distance of the eye of said patient; and

(b) selecting the lens such that its length is no greater than about 1. mm (preferably no greater than about 0.5 mm) larger than said white-to-white distance and its length is no smaller than about 1 mm less than said white-to-white distance.

In a preferred method, the white-to-white distance is measured by photographing the eye and measuring the white-to-white distance in the photograph using a measuring device calibrated to the scale of the photograph.

### BRIEF DESCRIPTION OF THE DRAWINGS

FIG. 1 is a schematic cut-away side view showing the placement of a posterior chamber phakic refractive lens in the eye.

FIG. 2 is a front view and cut-away side view of the eye showing the measurements used in the present invention.

FIG. 3 is a side view of a phakic refractive lens which can be used in the present invention.

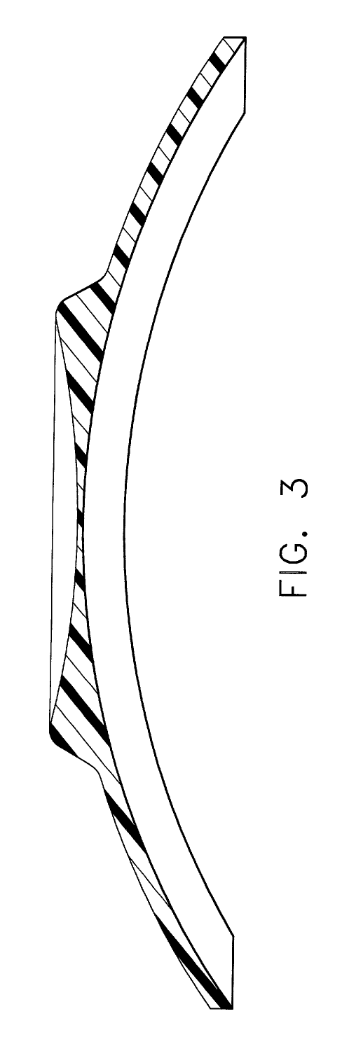

### DETAILED DESCRIPTION OF THE INVENTION

The object of the present invention is to use PRLs of appropriate design for correction of ametropia, such as myopia and hyperopia. It is also the object of the present invention to provide a standard method whereby the properly sized PRL is selected for an individual patient based on the patient's eye size. Specifically, the present invention relates to an easy and simple method for the measurement of the white-to-white distance of the eye as the standard for the determination of the overall length of the PRL suitable for the patient.

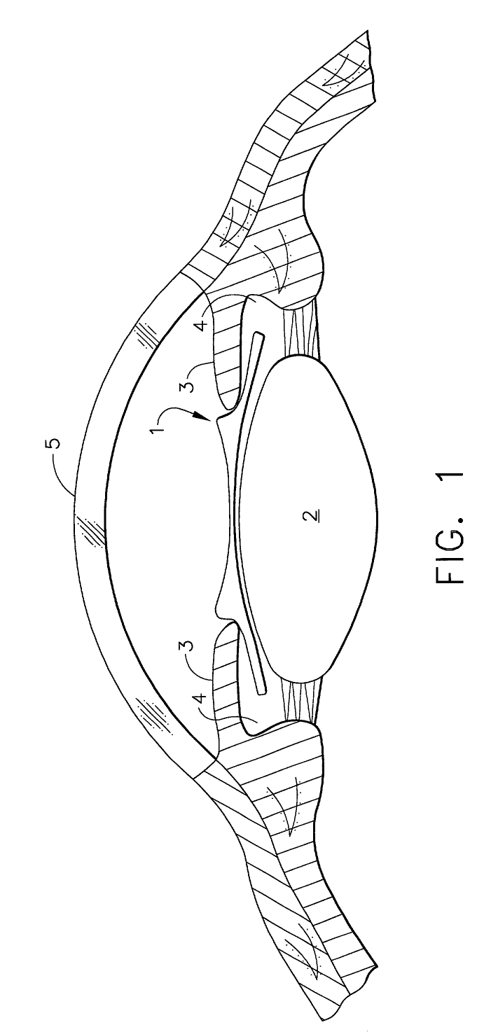

A floating PRL design does not have any kind of permanent fixation mechanism inside the eye. It allows the iris to move freely and constantly on its anterior surface without causing iris pigment dispersion. It also allows the aqueous humor to flow from anterior chamber to posterior chamber. In healthy eyes, this outflow occurs constantly. Blockage of this outflow of the aqueous humor will cause certain complications, such as intraocular pressure elevation, i.e., glaucoma. Because of these floating PRL design features, it is necessary that the PRL's length be approximately equal to or slightly less than the sulcus-to-sulcus diameter of a patient. An example of such a lens is shown in FIG. 1. In that figure, the PRL (1) is floating between the natural lens (2) and the iris (3). The cilliary sulcus is noted by (4). An oversized length will cause the PRL to arch or bend. This lens arching or bending will inevitably cause certain stress inside the eye, and it also hinders the free floating nature of the PRL when positioned in the posterior chamber. On the other hand, a PRL which is shorter than the sulcus-to-sulcus diameter will float when it is positioned inside the eye. However, if the PRL is too short, it may cause the PRL to decentralized inside the eye. The ideal length should be approximately equal to or slightly smaller than the sulcus-to-sulcus distance.

Those who are skilled in the art understand that modem imaging techniques can be used to precisely measure the sulcus-to-sulcus distance. One of them is high frequency echobiometry that can measure the sulcus-to-sulcus distance directly. Using high frequency echobiometry, PRL length can be determined precisely for a desirable fit in a given patient. However, high frequency echobiometry is a time consuming and costly procedure.

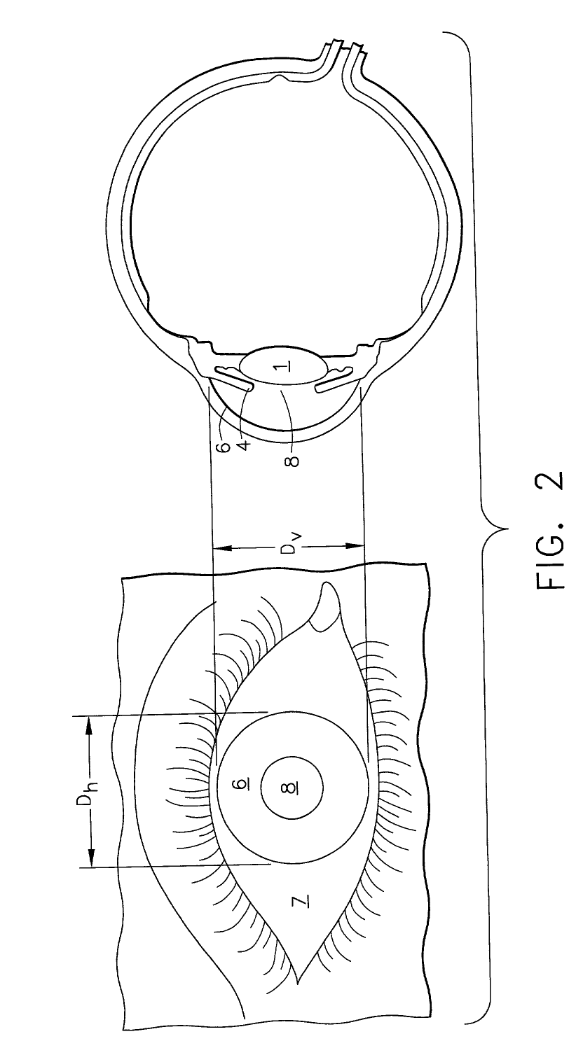

The present invention utilizes the white-to-white distance as the standard for the determination of the appropriate PRL size. The central part of the human cornea (5) is transparent to visible light. It appears clear. The clear cornea (6) has a circular or slightly oval shape and is located over the pupil (8). It is surrounded by sclera (7) which appears white (see FIG. 2). Because the clear cornea is not a perfectly circular shape, its diameter usually measured by a vertical white-to-white distance (Dᵥ) and a horizontal white-to-white distance (Dₕ). Patients' white-to-white distance varies, typically ranging from about 10 mm to about 13 mm. Those who are skilled in the art understand that the white-to-white distance can be measured in many different ways. For example, it can be directly measured by using a digital caliper closely above the cornea, but without touching it.

Alternatively, the white-to-white distance can be measured by a photographic method. A picture is taken of both the patient's eye and a ruler at the same magnification (such as 2×magnification). The patient's picture is usually taken using a slit lamp at the 2×magnification. The picture of the ruler is cut out so that a paper ruler with scales on it is obtained. The scale on the picture ruler can be calibrated with a digital caliper to ensure the correct conversion factor. After the calibration, the picture ruler works as a bio-meter for the picture of the patient. To measure the white-to-white distance, one simply lays the picture ruler over the picture of the patient's eye, then reads the number on the picture ruler. Once this procedure is completed, both the pictures of the eye and the ruler can be filed with the patient's medical history information.

After the patient's white-to-white distance is measured, the PRL's length can be determined. The ideal PRL length should be approximately equal to or (slightly) less than the white-to-white distance. If the horizontal white-to-white distance is slightly different than the vertical white-to-white distance, the larger distance is used for purposes of PRL size determination. Furthermore, our clinical experience indicates that the length (L) of PRL can be slightly bigger than the measured white-to-white distance, but should not exceed that distance by more than about 1 mm, preferably not more than about 0.5 mm. The length of the PRL should generally be no smaller than about 1 mm less (preferably no smaller than about 0.5 mm less) than the measured white-to-white distance. In summary, the ideal PRL length (L) for the floating lens design can be described as follows:

L≦Dₕ (or Dᵥ if it is larger than Dₕ)+1.0,

and

L≧Dₕ (or Dᵥ if it is larger than Dₕ)−2.0

Where:

L is the overall length (in mm) of the PRL

Dₕ is the horizontal white-to-white distance (in mm) (see FIG. 2)

Dᵥ is the vertical white-to-white distance (in mm) (see FIG. 2)

These equations can be used for both negative PRLs for the myopic patients and positive PRLs for the hyperopic patients.

Those who are skilled in the art will understand that the method described herein for determining the proper size of a PRL for a given patient, applies to a variety of lens designs including those which do not float in the eye. By following the teachings of the present application, there will be no arching or bending of the PRL inside the eye, caused by the mismatch of oversized PRL length with a relatively short sulcus-to-sulcus distance.

### EXAMPLES

### Example #1

The patient has a measured white-to-white distance of 11.7 mm (horizontal) and 11.2 mm (vertical). The patient has a BCVA (Best Corrected Visual Acuity) of 20/20 and BUVA (Best Uncorrected Visual Acuity) of worse than 20/200. The patient's spherical manifest refraction is −10.75 diopters. A PRL of −10 D with a length of 11.3 mm is implanted. The PRL is found to be able to rotate inside the eye. At the three-month post-operational examination, the patient's vision has been improved dramatically with BCVA of 20/10 and BUVA of 20/15. The patient's post-op spherical manifest refraction is +0.75 D.

### Example #2

The patient has a measured white-to-white distance of 12.9 mm (horizontal) and 12.0 mm (vertical). The patient has a BCVA of 20/20 and BUVA of worse than 20/200. The patient's spherical manifest refraction is −11.75 diopters. A PRL of −10 D with a length of 11.3 mm is implanted. The PRL was found to be able to move around inside the eye. At the one-month post-operational examination, the patient's vision has been improved dramatically with BCVA of 20/10 and BUVA of 20/20. No decentration of the PRL is found. The patient's post-op spherical manifest refraction is −0.5 D.

### Example #3

The patient has a measured white-to-white distance of 10.5 mm (horizontal) and 10.5 mm (vertical). The patient has a BCVA of 20/25 and BUVA of worse than 20/200. The patient's spherical manifest refraction is −14.75 diopters. A PRL of −12.0 D, with a length of 10.8 mm is implanted. There is no observation of lens arching due to the fact that the PRL is 0.3 mm longer than the white-to-white distance. At the one-month post-operational examination, the patient's vision has been improved dramatically with BCVA of 20/20 and BUVA of 20/20. The patient's spherical manifest refraction changed from the pre-operation of −14.75 to the post-op of 0. This means that the PRL's refraction power is perfectly selected for the patient.

### Example #4

The patient has a measured white-to-white distance of 12.0 mm (horizontal) and 11.9 mm (vertical). The patient has a BCVA of 20/20 and BUVA of 20/400. The patient's spherical manifest refraction is +6.75 diopters. A PRL of +8.0 D with a length of 10.6 mm is implanted. Although the PRL length is 1.4 mm shorter than the white-to-white distance, there was no observation of lens decentration inside the eye. At the three-month post-operational examination, the patient's vision has been improved dramatically with BCVA of 20/20 and BUVA of 20/30. The patient's spherical manifest refraction has changed from the pre-operation of +6.75 to the post-op of 0. This means the PRL's refraction power is perfectly selected for the patient.

What is claimed is:

1. A method for selecting a phakic intraocular lens for a given patient comprising the steps of: (a) measuring the white-to-white distance of the eye of said patient; and (b) selecting the lens such that its length is no greater than about 1 mm larger than said white-to-white distance and its length is no smaller than about 1 mm less than said white-to-white distance.

2. The method according to claim 1 wherein the length of the lens is no greater than about 0.5 mm larger than said white-to-white distance.

3. The method according to claim 2 where the length of the lens is no greater than said white-to-white distance.

4. The method according to claim 1 wherein the horizontal and vertical white-to-white distances are measured and the larger one is utilized to calculate the length of the lens.

5. The method according to claim 1 wherein the white-to-white distance is measured by photographing the eye and measuring the white-to-white distance in said photograph using a measuring device calibrated to the scale in said photograph.

6. The method of claim 5 wherein said measuring device is a photograph of a ruler calibrated to the scale in said photograph of the eye.

7. The method according to claim 1 wherein the phakic intraocular lens floats within the eye of the patient.

8. The method for selecting a phakic intraocular lens for a given patient comprising the steps of: measuring the horizontal and vertical distance of the eye of said patient; selecting the larger of the two distances; and selecting the lens such that its length is no greater than about 0.5 mm larger than said selected white-to-white distance and its length is no smaller than about 2 mm less than said selected white-to-white distance.
